(19) United States
(12) Patent Application Publication
(10) Pub. No.: US 2023/0226277 A1
(43) Pub. Date: Jul. 20, 2023

(54) SYSTEM FOR A DRUG DELIVERY DEVICE AND DRUG DELIVERY DEVICE

(71) Applicant: Sanofi, Paris (FR)

(72) Inventors: David Aubrey Plumptre, Worcestershire (GB); Robert Veasey, Warwick, Warwickshire (GB); Paul Griffin, Warwick, Warwickshire (GB); Rory James Livingston Hawkins, Warwick, Warwickshire (GB); Charles Henry Bertinat, Warwick, Warwick (GB)

(21) Appl. No.: 18/009,011

(22) PCT Filed: Jun. 18, 2021
(86) PCT No.: PCT/EP2021/066529

§ 371 Date: Dec. 8, 2022

(30) Foreign Application Priority Data
Jun. 19, 2020 (EP) 20315310.1

(51) Int. Cl. A61M 5/24

(52) U.S. Cl. CPC A61M 5/2422

(57) ABSTRACT

The present disclosure refers to a system for a drug delivery device comprising a housing and a cartridge holder which may contain a cartridge filled with a medicament. The housing and the cartridge holder comprise a coding interface for dedicating a certain cartridge to a certain housing and a bayonet type attachment interface for releasably attaching the cartridge holder to the housing by an attachment movement comprising a first stage and a second stage. The system further comprises at least one compliant spring member arranged and designed such that it is strained during the first and/or the second stage of the attachment movement at least if the housing coding element does not match the holder coding element, and wherein the compliant spring member is arranged and designed to move the cartridge holder relative to the housing in a direction contrary to the attachment movement when relaxing.

### CROSS REFERENCE TO RELATED APPLICATIONS

The present application is the national stage entry of International Patent Application No. PCT/EP2021/066529, filed on Jun. 18, 2021, and claims priority to Application No. EP 20315310.1, filed on Jun. 19, 2020, the disclosures of which are incorporated herein by reference.

### TECHNICAL FIELD

The present disclosure is generally directed to a system for a drug delivery device, e.g. an injection device for selecting and dispensing a number of user variable doses of a medicament. The disclosure is further directed to a drug delivery device, for example, reusable drug delivery devices, fixed dose devices which only allow dispensing of a predefined dose without the possibility to increase or decrease the set dose, etc.

### BACKGROUND

Pen type drug delivery devices have application where regular injection by persons without formal medical training occurs. This may be increasingly common among patients having diabetes where self-treatment enables such patients to conduct effective management of their disease. In practice, such a drug delivery device allows a user to individually select and dispense a number of user variable doses of a medicament.

There are basically two types of drug delivery devices: resettable devices (i.e., reusable) and non-resettable (i.e., disposable). For example, disposable pen delivery devices are supplied as self-contained devices. Such self-contained devices do not have removable pre-filled cartridges. Rather, the pre-filled cartridges may not be removed and replaced from these devices without destroying the device itself. Consequently, such disposable devices need not have a resettable dose setting mechanism.

In reusable devices, where a single drive mechanism which may be housed in a housing of the drug delivery device is used in conjunction with several cartridges or ampules to dispense drug contained in the cartridge or ampule from the device. An example of such a reusable drug delivery to which the present disclosure is applicable is known from WO 2014/033195 A1.

In such devices, usually a cartridge holder of the device is releasably connected or attached to the housing and can be removed or detached from the housing to replace a used cartridge. For doing so, the cartridge holder is disconnected from the housing, the used cartridge is removed from the holder and replaced with a new cartridge which is inserted into the cartridge holder, where the cartridge holder is again attached to the housing and the device is ready to be used again to dispense drug from the new cartridge. The system of the present disclosure comprises such a combination of a housing and a cartridge holder.

In such a system a range of dedicated cartridge holders may be used in conjunction with a set of different dedicated reusable pen injectors. Such dedication may be required for example where different medication cartridge sizes are used (e.g. 1.5 mL and 3 mL), or where the cartridges contain different concentrations of medication (e.g. U100 and U300). In this scenario a range of different pen injector mechanisms may be needed to deliver the medication because e.g. the piston rod travel per unit increment of medication will likely change for the different cartridge or medication types. Thus, systems of this kind have the risk that a cartridge containing a drug for which the mechanism of the drug delivery device is not specifically designed, i.e. a wrong drug, can be inserted into the cartridge holder and the user does not realize that he or she has put the wrong drug cartridge into the cartridge holder. This mistake may be lethal for the user and is also likely to occur as cartridges with different drugs may look highly similar.

To help to prevent a mix-up, a dedication system is known in which different cartridges are supplied to the market pre-assembled (irremovably) within dedicated cartridge holders. These dedicated cartridge holders are designed only to engage correctly (fully) with a compatible pen mechanism. Such a cartridge holder dedication system is described in WO 2020/016162 A1 disclosing a system comprising a housing and a cartridge holder with a bayonet type attachment interface for releasably attaching the cartridge holder to the housing and a coding interface that ensures that only matching pairs of housings and cartridge units can be connected.

For example, the system uses a common bayonet attachment geometry for all variants. In this system, dedication is achieved by having a range of different ramp feature geometries moulded onto the end surface of the cartridge holder and a range of corresponding different receiving features moulded into the housing, e.g. into an inner body component of the pen mechanism. If the ramps on the cartridge holder and inner body are compatible then the components will assemble correctly to the end location of the bayonet track. If however, the ramps are incompatible, then they will block full engagement of the cartridge holder bayonet.

A potential disadvantage of the dedication system described in WO 2020/016162 A1 may be that the bayonet features of the cartridge holder are permitted to partially engage with the bayonet tracks of the housing or inner body before the blocking occurs, and so in some circumstances the user may be confused as to whether the cartridge holder and pen mechanism are compatible or not.

US 2014/052075 A1 discloses a drug delivery device comprising a cartridge holder and a housing, wherein the cartridge holder is provided with a coding element which interacts with a counterpart of an interaction member which is not part of the housing. The interaction member needs to be rotatable with respect to the longitudinal axis of the device, i.e. with respect to the housing. This interaction member is merely a connecting element without any coding function.

WO 2008/074897 A1 discloses a container and a dosing assembly comprising a housing. The container comprises coding projections and the housing comprises a coding groove. The interaction of the coding projection and the coding groove ensures that only predetermined containers are fastened to corresponding dosing assemblies. The attachment of the container and the housing is realized by a fastening projection interacting with a fastening groove and a retaining projection interacting with a retaining groove. Further, the container is fastened to the assembly by an initial axial movement followed by a combined axial and rotational movement. For resetting of the device, a spring biases a coupling mechanism towards a distal position when no container is fastened to the housing. A push member biased by the spring exerts a distal force to a cartridge accommodated into a cartridge holder.

WO 2011/131777 A1 discloses a cartridge assembly configured to be connected to a dose setting mechanism depending of respective coding features. Further, the cartridge assembly and the dose setting mechanism comprise a groove configured to interact with a pin in order to fasten the cartridge assembly to the dosing mechanism if the coding features match, wherein the interaction comprises an axial and a rotational movement.

U.S. Pat. No. 9,687,612 B2 discloses a device with a dose setting mechanism and a cartridge assembly, wherein coding features are provided on a separate lock nut. The lock nut and therefore the coding elements are free to rotate with respect to the dose setting mechanism. In U.S. Pat. No. 9,687,612, the cartridge assembly can first be connected to the dispensing mechanism and only the function of the dispensing mechanism depends on the coding features.

### SUMMARY

This disclosure relates to an improved system and an improved drug delivery device providing additional, e.g. tactile or visual, feedback to the user when blocking occurs, such that it is more obvious when attachment of an incompatible cartridge holder/mechanism pairing is attempted.

According to one aspect of the present disclosure, a compliant spring member is located between the cartridge holder and the housing, i.e. the pen injector mechanism, that is loaded as the cartridge holder is attached to the pen injector. If an incompatible pairing is attempted, then blocking will occur, e.g. on the steep helical part of the bayonet track as a result of engagement between the incompatible ramp features. In this circumstance, the compliant spring located between the cartridge holder and the housing or pen injector mechanism will be loaded, applying a force and/or torque on to the cartridge holder that urges the cartridge holder back out of engagement. If the user releases their grip on the cartridge holder then the cartridge holder will, under the force/torque applied by the compliant spring member, move back up the bayonet track and away from the blocking location. This spring force, and the resultant movement of the cartridge holder when it is released, provides an indication to the user that the cartridge holder has not reached the correct attachment location and thus is not compatible with the pen injector mechanism.

According to a further aspect of the present disclosure, if the user attaches a cartridge holder that is compatible with the housing or pen injector mechanism then the compliant spring element between the cartridge holder and pen injector mechanism is configured such that it will either not be loaded during attachment, or any loading does not compromise the correct attachment or function of the pen. In other words, the cartridge holder may fully engage with the corresponding bayonet track of the mechanism. In the fully assembled location, the cartridge holder may be located in a region of the bayonet track where it is retained by a detent and there is preferably no resultant force that will cause it to disassemble from the pen mechanism.

In a general embodiment of the present disclosure, a system for use in a drug delivery device is provided, the system comprising a housing and a cartridge holder, wherein the housing comprises at least one housing coding element selected from a group of at least two different housing coding elements, and the cartridge holder comprises at least one holder coding element selected from a group of at least two different holder coding elements, and wherein the cartridge holder is attachable to the housing if the housing coding element provided on the housing matches with the holder coding element provided on the cartridge holder, whereas if the housing coding element provided on the housing does not match with the holder coding element provided on the cartridge holder full attachment of the cartridge holder to the housing is prevented. In addition, the housing and the cartridge holder comprise at least one, preferably bayonet type, attachment interface for releasably attaching the cartridge holder to the housing by an attachment movement, e.g. comprising a first stage and a second stage, wherein the first stage comprises at least an axial movement component of the cartridge holder relative to the housing and the second stage comprises at least a rotational movement component of the cartridge holder relative to the housing. Further, the system comprises at least one compliant, i.e. elastically deformable, spring member arranged and designed such that it is strained during the first and/or the second stage of the attachment movement at least if the housing coding element does not match the holder coding element. Still further, the compliant spring member is arranged and designed to move the cartridge holder relative to the housing in a direction contrary to the attachment movement when relaxing.

In other words, the compliant spring member may be part of a cartridge holder self-ejection mechanism causing or at least facilitating detachment of the cartridge holder from the housing and/or the pen injector mechanism if the cartridge holder and the housing are not compatible (housing coding element does not match the holder coding element). In addition, the compliant spring member may cause or facilitate detachment of the cartridge holder from the housing and/or the pen injector mechanism after rotating the cartridge holder relative to the housing in cases where the cartridge holder and the housing are compatible (housing coding element matches the holder coding element).

The present disclosure is not limited to a certain type, number or design of the at least one compliant spring member. Rather, there are various embodiments of suitable spring members as explained below.

For example, the at least one compliant spring member may be a spring washer arranged in the housing so as to exert a force on a cartridge if the cartridge is retained in the cartridge holder and the cartridge holder is fully attached to the housing. Thus, the spring washer may be designed and arranged suitable to secure the cartridge against axial movement with respect to the cartridge holder. The spring washer may comprise fixing elements, e.g. arms, extending in an axial direction for fixing the spring washer within the housing. This may include embodiments in which the fixing elements directly engage the housing and embodiments in which the fixing elements engage a sleeve or the like component part which in turn is retained in the housing.

The spring washer may be engaged by a counter element provided on the cartridge holder during the attachment movement. As an example for such a counter element, at least one rib may formed on an inner wall of the cartridge holder. The at least one rib is preferably located so that, if the housing coding element does not match the holder coding element, i.e. the housing and the cartridge holder are not compatible, at least at the end of the first stage of the attachment movement, an end surface of the at least one rib engages and compresses the spring washer. In more detail, the at least one rib may be formed on an inner wall of the cartridge holder. This rib is located so that when the blocking ramp features between the cartridge holder and pen mechanism are engaged, the end surface of the rib has engaged and slightly compressed the spring washer which applies a small axial force to the cartridge holder, which urges the part back out of engagement. On the other hand, if the user releases their grip on the cartridge holder, then the cartridge holder will, under the force applied by the spring washer, move back up the bayonet track and away from the blocking location.

This arrangement has the advantage that a spring element already existing in some pen injector mechanisms is used as the compliant spring member. Because the spring washer is a pressed metal component, it is not likely to be susceptible to creep or plastic deformation during the use life.

According to an aspect of the present disclosure, at least one holder coding element and at least one corresponding housing coding element may be arranged on the cartridge holder and on the housing, respectively. These coding elements are arranged on the the cartridge holder and on the housing, respectively, such that the at least one holder coding element and the at least one corresponding housing coding element may interact with each other during attachment of the cartridge holder on or in the housing. This interaction includes blocking of a full attachment of the cartridge holder on or in the housing in case of non-mating coding elements and permitting, e.g. guiding or at least not blocking, full attachment of the cartridge holder on or in the housing in case of non-mating coding elements. Thus, the location of the coding elements on the cartridge holder and on the housing, respectively, is such that at least non-mating coding elements may abut each other during attachment of the cartridge holder on or in the housing. For example, if the cartridge holder is to be inserted into the housing, the holder coding element may be arranged on an external surface of the cartridge holder and the corresponding housing coding element may be arranged on an internal surface of the housing. In addition or as an alternative, the holder coding element may be arranged on a proximally facing surface of the cartridge holder and the corresponding housing coding element may be arranged on a distally facing surface of the housing.

The present disclosure is not limited to a specific point in time during the attachment at which there is a contact of the coding elements compared to the engagement and movement of the bayonet interface. In case a full attachment of the cartridge holder to the housing is to be prevented if the coding elements do not match, non-matching coding elements may come into contact prior to engagement of the bayonet interface and/or during engagement of the bayonet interface. According to an aspect of the present disclosure, there may be two movements required for the attachment of the bayonet type attachment interfaces, namely an axial movement during the first stage and a rotational movement during the second stage. In this case, the coding elements that indicate whether the housing and the cartridge holder do match, may be in contact before, after and/or during the attachment movement of the bayonet type attachment interfaces, wherein the matching indication has to take place before a full attachment can be reached, i.e. before the end of the first and/or second stage of the attachment movement.

According to a further aspect of the present disclosure, the spring may be strained if the coding elements of the cartridge holder and the housing do not match. Thus, the spring may be strained independently of the matching of the housing and the cartridge holder and the present disclosure is not limited to straining the spring only if the housing and cartridge holder are compatible. For example, a relative movement between the cartridge holder and the housing may occur prior to a condition in which non-mating coding elements block further relative movements required for full attachment. This first relative movement may strain the spring. As an alternative or in addition, the spring may be preloaded.

According to an exemplary aspect of the present disclosure, a system for a drug delivery device may comprise a housing comprising:
at least one housing coding element selected from a group of at least two different housing coding elements, wherein the at least one housing coding element is arranged in or on the housing,
a cartridge holder comprising at least one holder coding element selected from a group of at least two different holder coding elements, wherein the at least one holder coding element is arranged in or on the cartridge holder,
wherein the housing and the cartridge holder comprise at least one bayonet type attachment interface for releasably attaching the cartridge holder to the housing by an attachment movement comprising a first stage and an, e.g. subsequent, second stage, wherein the first stage comprises at least an axial movement component of the cartridge holder relative to the housing and the second stage comprises at least a rotational movement component of the cartridge holder relative to the housing,
wherein the system further comprises at least one compliant spring member, e.g. a prestrained spring member, arranged and designed such that it is strained during the first and/or the second stage of the attachment movement at least if the housing coding element does not match the holder coding element, wherein the compliant spring member is arranged and designed to move the cartridge holder relative to the housing in a direction contrary to the attachment movement when relaxing,
and wherein the cartridge holder is releasably attachable to the housing if the housing coding element of the housing matches with the holder coding element of the cartridge holder, whereas if the housing coding element of the housing does not match with the holder coding element of a cartridge holder full attachment of the cartridge holder to the housing is prevented, i.e. only a portion or none of the first and/or the second stage of the attachment movement is permitted.

Preferably, the ribs are located on the bore of the cartridge holder such that they do not compress the spring washer when a cartridge holder is fully attached to a compatible pen injector mechanism, i.e. if the housing coding element provided on the housing matches with the holder coding element provided on the cartridge holder. This can be achieved because the rotational position of the cartridge holder with respect to the housing or pen mechanism is different when it is fully attached (to a compatible pen housing) compared with when it is in the blocked position (with an incompatible pen housing). In the fully attached location, the ribs of the cartridge holder do not coincide with the peaks of the spring washer and so the spring washer engages with the rim of the cartridge to keep it correctly biased during use.

According to an alternative example of the disclosure, the at least one compliant spring member comprises at least one compliant region of the cartridge holder which is, at least at the end of the first stage of the attachment movement, elastically deformable by engagement of the housing coding element and the holder coding element if the housing coding element does not match the holder coding element. In other words, the second embodiment of the disclosure uses compliant regions of the cartridge holder moulding to store and release torque to move the cartridge holder back up the bayonet track, away from the blocking location when the user releases their grip. These compliant regions may be created by one or more slots that may be formed at the end of the cartridge holder. Thus, the at least one compliant region of the cartridge holder may comprise an axially extending slot in the cartridge holder. As an alternative, the at least one compliant region of the cartridge holder may comprise a circumferentially extending slot in the cartridge holder.

In more detail, when torque is applied by the user to attempt to screw the cartridge holder past the blocking location, this may be reacted as a force/torque between the, e.g. ramp shaped, housing coding element provided on the housing and the, e.g. ramp shaped, holder coding element provided on the cartridge holder. Owing to the slots formed in the end of the cartridge holder component, this component is somewhat elastically compliant and acts as a spring under the user applied torque. When the user releases the cartridge holder in the blocked location, any energy stored in the cartridge holder by its' elastic deflection will be released, causing it to unwind a small distance back up the bayonet track.

In a still further example of the present disclosure, the at least one compliant spring member may comprise at least one compression spring having a distal end (facing to the needle end of the device) attached to or abutting the cartridge holder. The compression spring may be, at least at the end of the first stage of the attachment movement, elastically deformable by engagement of its opposite proximal end with the housing or a component retained in the housing. The at least one compression spring is preferably configured as either a metal wire-formed compression spring, a metal pressing or a plastic moulding. The present disclosure is not limited to embodiments in which this compression spring is compressed only if the housing and the cartridge holder are not compatible with each other but is also applicable when the compression spring is strained irrespective of the compatibility of the housing and the cartridge holder.

In more detail, the compression spring may be, at least at the end of the first stage of the attachment movement, elastically deformable by engagement of its opposite proximal end with a spring washer or a bias spring element as the component retained in the housing. Preferably, this spring washer is arranged in the housing so as to exert a force on a cartridge if the cartridge is retained in the cartridge holder and the cartridge holder is fully attached to the housing. Thus, the spring washer may be designed and arranged suitable to secure the cartridge against axial movement with respect to the cartridge holder. The spring washer may comprise fixing elements, e.g. arms, extending in an axial direction for fixing the spring washer within the housing. This may include embodiments in which the fixing elements directly engage the housing and embodiments in which the fixing elements engage a sleeve or the like component part which in turn is retained in the housing.

Preferably, the stiffness of the spring washer is higher than the stiffness of the at least one compression spring. If the compression spring in the cartridge holder assembly is not stiff enough to cause significant deflection of the spring washer located in the pen mechanism, the compression spring member will be deflected by contact with the spring washer owing to its much lower stiffness. This means that the spring washer is still able to perform its key function of engaging and biasing the cartridge forwards onto its datum contact surface with the cartridge holder.

In the system according to the present disclosure, the housing coding element and the holder coding element preferably form a ramp interface. The second stage of the attachment movement may be defined or governed by this ramp interface and/or by the bayonet type attachment interface. In an example, the bayonet type attachment interface is configured such that the cartridge holder is moved relative to the housing during the second stage of the attachment movement an axial distance in a second stage axial direction which is opposite to the first stage axial direction when the cartridge holder is attached to the housing. Preferably, matching of the housing coding element provided on the housing with the holder coding element provided on the cartridge holder, i.e. compatibility of the housing and the cartridge holder, is defined by a slope of the ramp interface being equal to a slope defined by the second stage axial distance.

The design, function and arrangement of the interfaces between the housing and the cartridge holder, with the exception of the at least one compliant spring member of the present disclosure, is preferably as disclosed in WO 2020/016162 A1 to which reference is made for the description of these interfaces.

In an embodiment, the cartridge holder is part of a cartridge assembly. The assembly may comprise the cartridge and the cartridge holder. The cartridge may be permanently and/or irreleasably secured in the cartridge holder. Thus, the cartridge unit may form a disposable item. Alternatively, the cartridge may be releasably secured in the cartridge holder.

The drug delivery device may comprise a dosing section or dose setting mechanism which is typically the portion of the pen device that is used to set (select) a dose. During an injection, a spindle or piston rod contained within the dose setting mechanism presses against the bung or stopper of the cartridge. This force causes the medication contained within the cartridge to be injected through an attached needle assembly. After an injection, as generally recommended by most drug delivery device and/or needle assembly manufacturers and suppliers, the needle assembly is removed and discarded.

The present disclosure is applicable for devices which are manually driven, e.g. by a user applying a force to an injection button, for devices which are driven by a spring or electric motor or the like and for devices which combine these two concepts, i.e. power assisted devices which still require a user to exert an injection force. The spring-type devices involve springs which are preloaded and springs which are loaded by the user during dose selecting. Some stored-energy devices use a combination of spring preload and additional energy provided by the user, for example during dose setting.

The present disclosure further pertains to a drug delivery device comprising a system as described above, the drug delivery device comprising a medicament.

The terms “drug” or “medicament” are used synonymously herein and describe a pharmaceutical formulation containing one or more active pharmaceutical ingredients or pharmaceutically acceptable salts or solvates thereof, and optionally a pharmaceutically acceptable carrier. An active pharmaceutical ingredient (“API”), in the broadest terms, is a chemical structure that has a biological effect on humans or animals. In pharmacology, a drug or medicament is used in the treatment, cure, prevention, or diagnosis of disease or used to otherwise enhance physical or mental well-being. A drug or medicament may be used for a limited duration, or on a regular basis for chronic disorders.

As described below, a drug or medicament can include at least one API, or combinations thereof, in various types of formulations, for the treatment of one or more diseases. Examples of API may include small molecules having a molecular weight of 500 Da or less; polypeptides, peptides and proteins (e.g., hormones, growth factors, antibodies, antibody fragments, and enzymes); carbohydrates and polysaccharides; and nucleic acids, double or single stranded DNA (including naked and cDNA), RNA, antisense nucleic acids such as antisense DNA and RNA, small interfering RNA (siRNA), ribozymes, genes, and oligonucleotides. Nucleic acids may be incorporated into molecular delivery systems such as vectors, plasmids, or liposomes. Mixtures of one or more drugs are also contemplated.

The drug or medicament may be contained in a primary package or “drug container” adapted for use with a drug delivery device. The drug container may be, e.g., a cartridge, syringe, reservoir, or other solid or flexible vessel configured to provide a suitable chamber for storage (e.g., short- or long-term storage) of one or more drugs. For example, in some instances, the chamber may be designed to store a drug for at least one day (e.g., 1 to at least 30 days). In some instances, the chamber may be designed to store a drug for about 1 month to about 2 years. Storage may occur at room temperature (e.g., about 20° C.), or refrigerated temperatures (e.g., from about −4° C. to about 4° C.). In some instances, the drug container may be or may include a dual-chamber cartridge configured to store two or more components of the pharmaceutical formulation to-be-administered (e.g., an API and a diluent, or two different drugs) separately, one in each chamber. In such instances, the two chambers of the dual-chamber cartridge may be configured to allow mixing between the two or more components prior to and/or during dispensing into the human or animal body. For example, the two chambers may be configured such that they are in fluid communication with each other (e.g., by way of a conduit between the two chambers) and allow mixing of the two components when desired by a user prior to dispensing. Alternatively or in addition, the two chambers may be configured to allow mixing as the components are being dispensed into the human or animal body.

The drugs or medicaments contained in the drug delivery devices as described herein can be used for the treatment and/or prophylaxis of many different types of medical disorders. Examples of disorders include, e.g., diabetes mellitus or complications associated with diabetes mellitus such as diabetic retinopathy, thromboembolism disorders such as deep vein or pulmonary thromboembolism. Further examples of disorders are acute coronary syndrome (ACS), angina, myocardial infarction, cancer, macular degeneration, inflammation, hay fever, atherosclerosis and/or rheumatoid arthritis. Examples of APIs and drugs are those as described in handbooks such as Rote Liste 2014, for example, without limitation, main groups 12 (anti-diabetic drugs) or 86 (oncology drugs), and Merck Index, 15th edition.

Examples of APIs for the treatment and/or prophylaxis of type 1 or type 2 diabetes mellitus or complications associated with type 1 or type 2 diabetes mellitus include an insulin, e.g., human insulin, or a human insulin analogue or derivative, a glucagon-like peptide (GLP-1), GLP-1 analogues or GLP-1 receptor agonists, or an analogue or derivative thereof, a dipeptidyl peptidase-4 (DPP4) inhibitor, or a pharmaceutically acceptable salt or solvate thereof, or any mixture thereof. As used herein, the terms “analogue” and “derivative” refers to a polypeptide which has a molecular structure which formally can be derived from the structure of a naturally occurring peptide, for example that of human insulin, by deleting and/or exchanging at least one amino acid residue occurring in the naturally occurring peptide and/or by adding at least one amino acid residue. The added and/or exchanged amino acid residue can either be codeable amino acid residues or other naturally occurring residues or purely synthetic amino acid residues. Insulin analogues are also referred to as “insulin receptor ligands”. In particular, the term “derivative” refers to a polypeptide which has a molecular structure which formally can be derived from the structure of a naturally occurring peptide, for example that of human insulin, in which one or more organic substituent (e.g. a fatty acid) is bound to one or more of the amino acids. Optionally, one or more amino acids occurring in the naturally occurring peptide may have been deleted and/or replaced by other amino acids, including non-codeable amino acids, or amino acids, including non-codeable, have been added to the naturally occurring peptide. Examples of insulin analogues are Gly(A21), Arg(B31), Arg(B32) human insulin (insulin glargine); Lys(B3), Glu(B29) human insulin (insulin glulisine); Lys(B28), Pro(B29) human insulin (insulin lispro); Asp(B28) human insulin (insulin aspart); human insulin, wherein proline in position B28 is replaced by Asp, Lys, Leu, Val or Ala and wherein in position B29 Lys may be replaced by Pro; Ala(B26) human insulin; Des(B28-B30) human insulin; Des(B27) human insulin and Des(B30) human insulin.

Examples of insulin derivatives are, for example, B29-N-myristoyl-des(B30) human insulin, Lys(B29) (N-tetradecanoyl)-des(B30) human insulin (insulin detemir, Levemir®); B29-N-palmitoyl-des(B30) human insulin; B29-N-myristoyl human insulin; B29-N-palmitoyl human insulin; B28-N-myristoyl LysB28ProB29 human insulin; B28-N-palmitoyl-LysB28ProB29 human insulin; B30-N-myristoyl-ThrB29LysB30 human insulin; B30-N-palmitoyl-ThrB29LysB30 human insulin; B29-N-(N-palmitoyl-gamma-glutamyl)-des(B30) human insulin, B29-N-omega-carboxypentadecanoyl-gamma-L-glutamyl-des(B30) human insulin (insulin degludec, Tresiba®); B29-N-(N-lithocholyl-gamma-glutamyl)-des(B30) human insulin; B29-N-(ω-carboxyheptadecanoyl)-des(B30) human insulin and B29-N-(ω-carboxyheptadecanoyl) human insulin.

Examples of GLP-1, GLP-1 analogues and GLP-1 receptor agonists are, for example, Lixisenatide (Lyxumia®), Exenatide (Exendin-4, Byetta®, Bydureon®, a 39 amino acid peptide which is produced by the salivary glands of the Gila monster), Liraglutide (Victoza®), Semaglutide, Taspoglutide, Albiglutide (Syncria®), Dulaglutide (Trulicity®), rExendin-4, CJC-1134-PC, PB-1023, TTP-054, Langlenatide/HM-11260C (Efpeglenatide), HM-15211, CM-3, GLP-1 Eligen, ORMD-0901, NN-9423, NN-9709, NN-9924, NN-9926, NN-9927, Nodexen, Viador-GLP-1, CVX-096, ZYOG-1, ZYD-1, GSK-2374697, DA-3091, MAR-701, MAR709, ZP-2929, ZP-3022, ZP-DI-70, TT-401 (Pegapamodtide), BHM-034. MOD-6030, CAM-2036, DA-15864, ARI-2651, ARI-2255, Tirzepatide (LY3298176), Bamadutide (SAR425899), Exenatide-XTEN and Glucagon-Xten.

An example of an oligonucleotide is, for example: mipomersen sodium (Kynamro®), a cholesterol-reducing antisense therapeutic for the treatment of familial hypercholesterolemia or RG012 for the treatment of Alport syndrome.

Examples of DPP4 inhibitors are Linagliptin, Vildagliptin, Sitagliptin, Denagliptin, Saxagliptin, Berberine.

Examples of hormones include hypophysis hormones or hypothalamus hormones or regulatory active peptides and their antagonists, such as Gonadotropine (Follitropin, Lutropin, Choriongonadotropin, Menotropin), Somatropine (Somatropin), Desmopressin, Terlipressin, Gonadorelin, Triptorelin, Leuprorelin, Buserelin, Nafarelin, and Goserelin.

Examples of polysaccharides include a glucosaminoglycane, a hyaluronic acid, a heparin, a low molecular weight heparin or an ultra-low molecular weight heparin or a derivative thereof, or a sulphated polysaccharide, e.g. a poly-sulphated form of the above-mentioned polysaccharides, and/or a pharmaceutically acceptable salt thereof. An example of a pharmaceutically acceptable salt of a poly-sulphated low molecular weight heparin is enoxaparin sodium. An example of a hyaluronic acid derivative is Hylan G-F 20 (Synvisc®), a sodium hyaluronate.

The term “antibody”, as used herein, refers to an immunoglobulin molecule or an antigen-binding portion thereof. Examples of antigen-binding portions of immunoglobulin molecules include F(ab) and F(ab′)2 fragments, which retain the ability to bind antigen. The antibody can be polyclonal, monoclonal, recombinant, chimeric, de-immunized or humanized, fully human, non-human, (e.g., murine), or single chain antibody. In some embodiments, the antibody has effector function and can fix complement. In some embodiments, the antibody has reduced or no ability to bind an Fc receptor. For example, the antibody can be an isotype or subtype, an antibody fragment or mutant, which does not support binding to an Fc receptor, e.g., it has a mutagenized or deleted Fc receptor binding region. The term antibody also includes an antigen-binding molecule based on tetravalent bispecific tandem immunoglobulins (TBTI) and/or a dual variable region antibody-like binding protein having cross-over binding region orientation (CODV).

The terms “fragment” or “antibody fragment” refer to a polypeptide derived from an antibody polypeptide molecule (e.g., an antibody heavy and/or light chain polypeptide) that does not comprise a full-length antibody polypeptide, but that still comprises at least a portion of a full-length antibody polypeptide that is capable of binding to an antigen. Antibody fragments can comprise a cleaved portion of a full length antibody polypeptide, although the term is not limited to such cleaved fragments. Antibody fragments that are useful in the present disclosure include, for example, Fab fragments, F(ab′)2 fragments, scFv (single-chain Fv) fragments, linear antibodies, monospecific or multispecific antibody fragments such as bispecific, trispecific, tetraspecific and multispecific antibodies (e.g., diabodies, triabodies, tetrabodies), monovalent or multivalent antibody fragments such as bivalent, trivalent, tetravalent and multivalent antibodies, minibodies, chelating recombinant antibodies, tribodies or bibodies, intrabodies, nanobodies, small modular immunopharmaceuticals (SMIP), binding-domain immunoglobulin fusion proteins, camelized antibodies, and VHH containing antibodies. Additional examples of antigen-binding antibody fragments are known in the art.

The terms “Complementarity-determining region” or “CDR” refer to short polypeptide sequences within the variable region of both heavy and light chain polypeptides that are primarily responsible for mediating specific antigen recognition. The term “framework region” refers to amino acid sequences within the variable region of both heavy and light chain polypeptides that are not CDR sequences, and are primarily responsible for maintaining correct positioning of the CDR sequences to permit antigen binding. Although the framework regions themselves typically do not directly participate in antigen binding, as is known in the art, certain residues within the framework regions of certain antibodies can directly participate in antigen binding or can affect the ability of one or more amino acids in CDRs to interact with antigen. Examples of antibodies are anti PCSK-9 mAb (e.g., Alirocumab), anti IL-6 mAb (e.g., Sarilumab), and anti IL-4 mAb (e.g., Dupilumab).

Pharmaceutically acceptable salts of any API described herein are also contemplated for use in a drug or medicament in a drug delivery device. Pharmaceutically acceptable salts are for example acid addition salts and basic salts.

Those of skill in the art will understand that modifications (additions and/or removals) of various components of the APIs, formulations, apparatuses, methods, systems and embodiments described herein may be made without departing from the full scope and spirit of the present invention, which encompass such modifications and any and all equivalents thereof.

An example drug delivery device may involve a needle-based injection system as described in Table 1 of section 5.2 of ISO 11608-1:2014(E). As described in ISO 11608-1:2014(E), needle-based injection systems may be broadly distinguished into multi-dose container systems and single-dose (with partial or full evacuation) container systems. The container may be a replaceable container or an integrated non-replaceable container.

As further described in ISO 11608-1:2014(E), a multi-dose container system may involve a needle-based injection device with a replaceable container. In such a system, each container holds multiple doses, the size of which may be fixed or variable (pre-set by the user). Another multi-dose container system may involve a needle-based injection device with an integrated non-replaceable container. In such a system, each container holds multiple doses, the size of which may be fixed or variable (pre-set by the user).

As further described in ISO 11608-1:2014(E), a single-dose container system may involve a needle-based injection device with a replaceable container. In one example for such a system, each container holds a single dose, whereby the entire deliverable volume is expelled (full evacuation). In a further example, each container holds a single dose, whereby a portion of the deliverable volume is expelled (partial evacuation). As also described in ISO 11608-1:2014(E), a single-dose container system may involve a needle-based injection device with an integrated non-replaceable container. In one example for such a system, each container holds a single dose, whereby the entire deliverable volume is expelled (full evacuation). In a further example, each container holds a single dose, whereby a portion of the deliverable volume is expelled (partial evacuation).

The terms “axial”, “radial”, or “circumferential” as used herein may be used with respect to a main longitudinal axis of the device, the cartridge, the housing or the cartridge holder, e.g. the axis which extends through the proximal and distal ends of the cartridge, the cartridge holder or the drug delivery device.

Non-limiting, exemplary embodiments of the disclosure will now be described with reference to the accompanying drawings, in which:

### BRIEF DESCRIPTION OF THE FIGURES

FIG. 1 shows detailed section views of a range of dedicated cartridge holder components (only one of which is compatible with the housing component);

FIG. 2 shows a known cartridge holder;

FIG. 3 shows a section through a system of a first embodiment of the present disclosure, in which the cartridge holder is located in the blocking engagement position with respect to an incompatible housing;

FIG. 4 shows the cartridge holder of a second embodiment, showing compliant regions formed by slots;

FIG. 5 is an enlarged sectional view of the cartridge holder of the second embodiment;

FIG. 6 shows the cartridge holder of a third embodiment, showing compliant regions formed by slots;

FIG. 7 is an enlarged sectional view of the cartridge holder of the third embodiment;

FIG. 8 shows the cartridge holder of a fourth embodiment containing a compression spring;

FIG. 9 is a partially enlarged sectional view through the system of the fourth embodiment, in which the cartridge holder is located in the blocking engagement position with respect to an incompatible housing; and

FIG. 10 is a partially enlarged sectional view through the system of the fourth embodiment, in which the cartridge holder is located in the fully engaged position with respect to a compatible housing.

### DETAILED DESCRIPTION

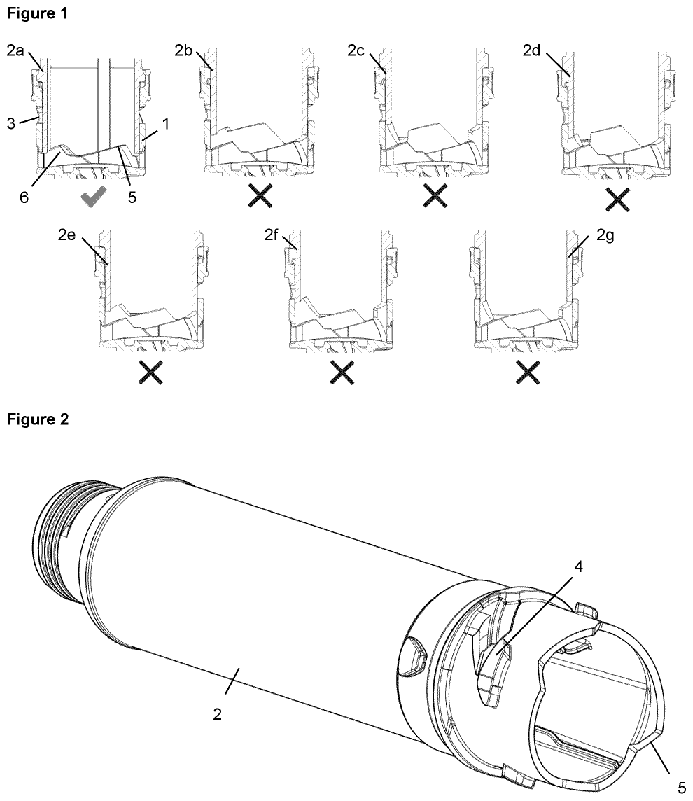

Referring to FIG. 1, a cartridge holder dedication system is shown which is based on the principle described in WO 2020/016162 A1 to which reference is made. This system comprises the combination of a pen injector mechanism housing 1 and a cartridge holder 2 which comprise interfaces adapted to each other. The housing 1 may be an outer housing or an inner housing of a drug delivery device. The cartridge holder may comprise a cartridge (not shown in FIG. 1) filled with a medicament. There may be a set of similar cartridge holders 2a to 2g and a set of similar housings 1 (only one is shown in FIG. 1) with the interfaces adapted such that only a certain cartridge holder 2a fits into and is compatible with a certain housing 1. The other cartridge holders 2b to 2g are not compatible with housing 1 as indicated by the “X” in FIG. 1.

The example shown in FIG. 1 is based on the use of a common bayonet attachment geometry for all variants, i.e. the housing 1 has a bayonet feature, here a bayonet slot or track 3, and all cartridge holders 2a to 2g have the identical mating counter bayonet feature, here bayonet protrusions 4 or keys (see FIGS. 2, 4, 6 and 8). The bayonet track 3 has at least two distinct portions, namely a first steep helical part of the bayonet track and a second, e.g. subsequent, flatter helical part of the bayonet track. As a consequence, an attachment movement of the cartridge holder 2a to 2g relative to the housing 1 comprises a first stage and a second stage, with the first stage comprising more axial than rotational movement of the bayonet protusions 4 in the first steep helical part of the bayonet track 3 and the second stage comprising a more rotational than axial movement of the bayonet protusions 4 in the second flatter helical part of the bayonet track 3. The slope of the second flatter helical part of the bayonet track 3 may be such that during the second stage the bayonet protusions 4 are slightly moved in an opposite axial direction compared with the first stage movement. In any case the first stage comprises at least an axial movement component of the cartridge holder 2a to 2g relative to the housing 1 and the second stage comprises at least a rotational movement component of the cartridge holder relative to the housing.

In this system, dedication is achieved by having a range of different coding elements in the form of ramp feature geometries moulded onto the end surface of the cartridge holder component 2a to 2g (holder coding element 5) and a range of corresponding different receiving features (housing coding element 6) moulded into the housing component 1 of the pen mechanism. If the ramps 5, 6 on the cartridge holder 2a to 2g and on the housing 1 are compatible then the components will assemble correctly to the end location of the bayonet track 3. If however, the ramps 5, 6 are incompatible, then they will block full engagement of the cartridge holder bayonet protrusions 4, as shown in FIG. 1.

As can be seen in FIG. 1, the bayonet protrusions 4 of the cartridge holder 2a to 2g are permitted to partially engage with the bayonet tracks 3 of the housing 1 before the blocking occurs, and so in some circumstances the user may be confused as to whether the cartridge holder 2a to 2g and pen mechanism housing 1 are compatible or not.

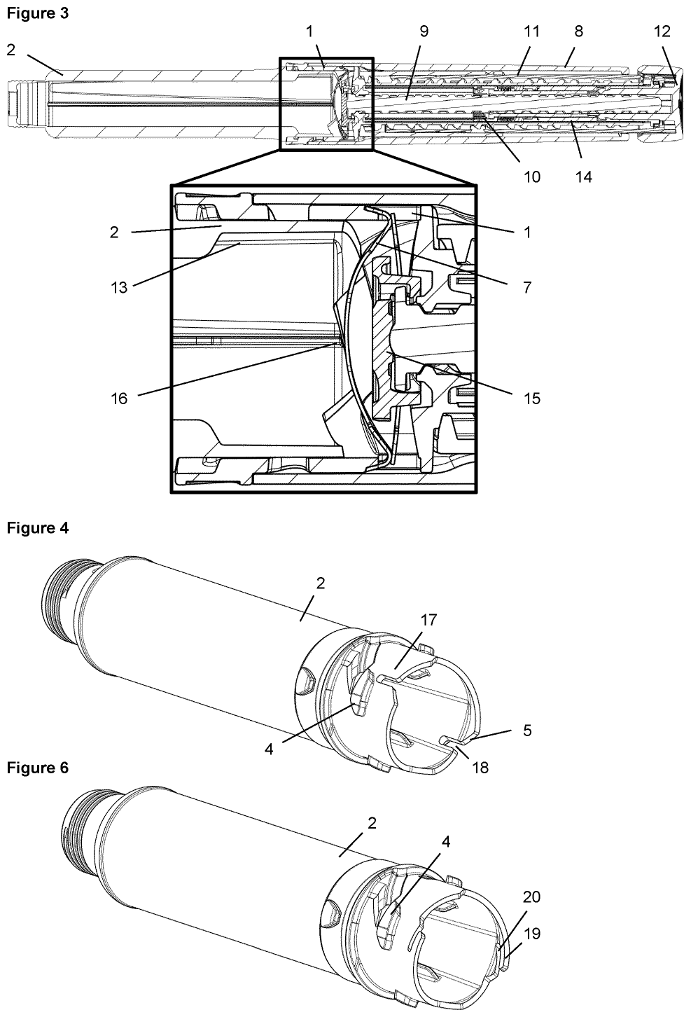

The present disclosure as depicted in FIGS. 3 to 10 provides additional tactile and visual feedback to the user when blocking occurs, such that it is more obvious when attachment of an incompatible cartridge holder/housing pairing is attempted. This is achieved by locating a compliant spring member (described below in more detail) between the cartridge holder 2a to 2g and pen injector mechanism housing 1 that is loaded as the cartridge holder 2a to 2g is attached to the pen injector.

If an incompatible pairing is attempted, then blocking will occur on the steep helical part of the bayonet track 3 as a result of engagement between the incompatible ramp features 5, 6. In this circumstance, the compliant spring located between the cartridge holder 2a to 2g and the pen injector mechanism housing 1 will be loaded, applying a force and/or torque on to the cartridge holder 2a to 2g that urges the cartridge holder back out of engagement.

If the user releases their grip on the cartridge holder 2a to 2g then the cartridge holder will, under the force/torque applied by the compliant spring member, move back up the bayonet track 3 and away from the blocking location. This spring force, and the resultant movement of the cartridge holder 2a to 2g when it is released, provides an indication to the user that the cartridge holder 2a to 2g has not reached the correct attachment location and thus is not compatible with the pen injector mechanism housing 1.

If the user attaches a cartridge holder 2a that is compatible with a pen injector mechanism housing 1 then the compliant spring element between the cartridge holder and pen injector mechanism housing is configured such that it will either not be loaded during attachment, or any loading does not compromise the correct attachment or function of the pen. The cartridge holder 2a engages fully with the corresponding bayonet track 3 of the mechanism. In the fully assembled location, the cartridge holder 2a is preferably located in a region of the bayonet track 3 where it is retained by a detent and there is no resultant force that will cause it to disassemble from the pen mechanism.

The present disclosure is applicable to various types of drug delivery devices. As a non limiting example, a drug delivery device of the type known from WO 2014/033195 A1 is shown in FIG. 3. This device has a distal end (left end in FIG. 3) and a proximal end (right end in FIG. 3). The drug delivery device comprises an outer housing part 8, a spring washer 7, the housing 1 which is an inner body in this example, a piston rod 9, a driver 10, a display member or number sleeve 11, a button 12, the cartridge holder 2 for receiving a cartridge 13, a clutch 14 and a pressure foot 15.

FIG. 3 shows a first embodiment of the present disclosure, which uses the spring washer 7 to provide the compliant spring member. In known devices such a spring washer 7 may be provided as a pressed metal cartridge bias spring to hold a cartridge 13 in a predefined position with respect to the cartridge holder 2. These spring washers 7 may be located directly or indirectly fixed within the pen injector mechanism housing, e.g. by means of axially extending fixing elements. For example, in FIG. 3 the spring washer 7 is fixed to the outer housing 8 via the inner housing 1. FIG. 3 shows a section through the drug delivery device assembly in which the cartridge holder 2 is located in the blocking engagement position with respect to an incompatible pen injector mechanism housing 1.

The enlarged lower image of FIG. 3 shows a region of the sectional view of the upper image. In the enlarged view it can be seen that at least one rib 16 is formed on the inner wall of the cartridge holder component 2. This rib 16 is located so that when the blocking ramp features 5, 6 between the cartridge holder 2 and pen mechanism housing 1 are engaged, the proximal end surface of the rib 16 has engaged and slightly compressed the spring washer 7. This applies a small axial force to the cartridge holder 2, which urges the part back out of engagement. If the user releases their grip on the cartridge holder 2, then the cartridge holder 2 will, under the force applied by the spring washer 7, move back up the bayonet track 3 and away from the blocking location.

This arrangement has the advantage that the existing spring washer 7 in the pen injector mechanism is used as the compliant spring member. Because the spring is a pressed metal component, it is not likely to be susceptible to creep or plastic deformation during the use life.

The ribs 16 are located in or on the bore of the cartridge holder 2 such that they do not compress the spring washer 7 when a cartridge holder 2 is fully attached to a compatible pen injector mechanism housing. This can be achieved because the rotational position of the cartridge holder 2 with respect to the pen mechanism is different when it is fully attached (to a compatible pen) compared with when it is in the blocked position (with an incompatible pen). In the fully attached location, the ribs 16 of the cartridge holder 2 do not coincide with the peaks of the spring washer 7 in the pen and so the spring washer 7 engages with the rim of the cartridge 13 to keep it correctly biased during use.

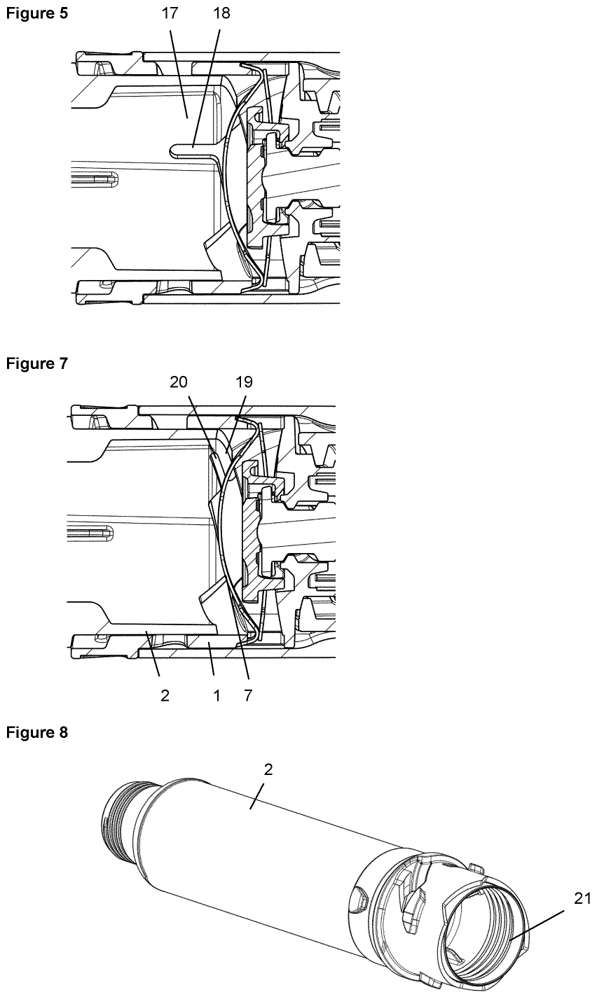

FIGS. 4 and 5 show a second embodiment of the present disclosure which uses compliant regions 17 of the cartridge holder moulding to store and release torque to move the cartridge holder 2 back up the bayonet track 3, away from the blocking location when the user releases their grip. These compliant regions 17 are created by slots 18 that are formed at the proximal end of the cartridge holder 2. In FIGS. 4 and 5, the slots 18 extend mainly axially.

As can be seen in FIG. 5, when torque is applied by the user to attempt to screw the cartridge holder 2 past the blocking location, this is reacted as a force/torque between the ramps 5 of the cartridge holder 2 and the corresponding features 6 of the inner housing 1. Owing to the slots 18 formed in the end of the cartridge holder 2, this component is somewhat elastically compliant and acts as a spring under the user applied torque. When the user releases the cartridge holder 2 in the blocked location, any energy stored in the cartridge holder 2 by its' elastic deflection will be released, causing it to unwind a small distance back up the bayonet track 3.

FIGS. 6 and 7 show a third embodiment of the present disclosure, which uses differently configured compliant elements 19 of the cartridge holder moulding to provide the force which moves the cartridge holder 2 back up the bayonet track 3, away from the blocking location when the user releases their grip. These compliant elements 19 are created in the ramp features 5 of the cartridge holder 5 by the introduction of slots 20 that are formed in the part.

As can be seen in FIG. 7, when torque is applied by the user to attempt to screw the cartridge holder 2 past the blocking location, this is reacted as a force on the compliant elements 19 formed by the slots 20 in the end of the cartridge holder 2. The compliant elements 19 are elastically compliant and act as cantilever type springs under the user applied torque. When the user releases the cartridge holder 2 in the blocked location, any energy stored in the compliant elements 19 of the cartridge holder 2 by its' elastic deflection will be released, causing it to unwind a small distance back up the bayonet track 3.

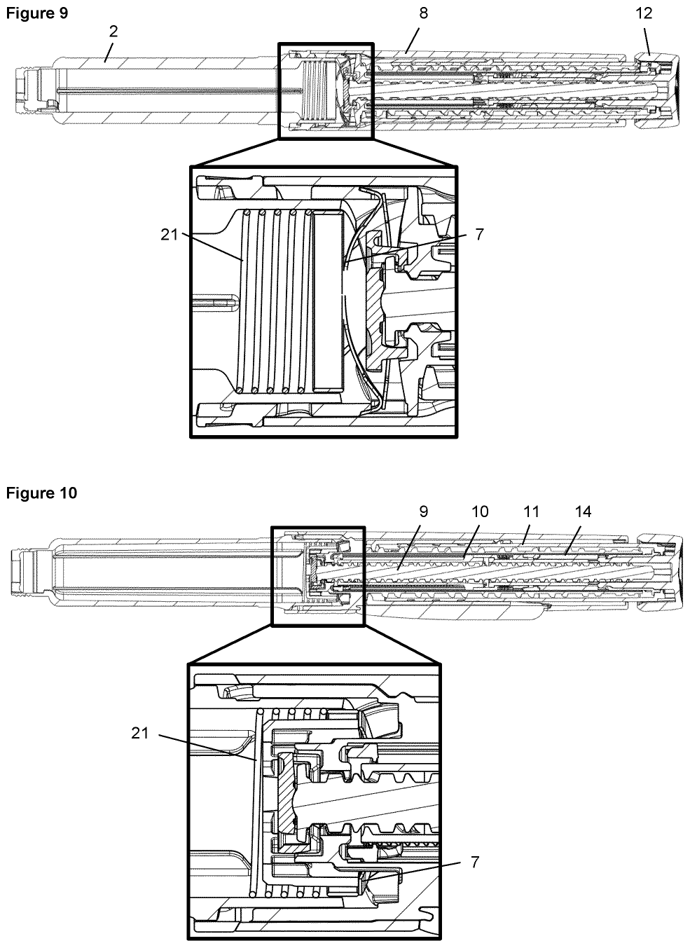

FIGS. 8 to 10 show a fourth embodiment of the present disclosure, which uses a separate additional spring member 21 to provide force that moves the cartridge holder 2 back up the bayonet track 3, away from the blocking location when the user releases their grip. The spring member 21 is a part of the cartridge holder assembly and can be configured as a metal wire-formed compression spring. As an alternative, the spring member may be a metal pressing or a plastic moulding (not shown). FIG. 8 shows a cartridge holder assembly of the fourth embodiment of the disclosure, in which a wire-formed compression spring 21 is located in the proximal end of the assembly. FIGS. 9 and 10 both show the identical drug delivery device but with different cartridge holders. In addition, FIG. 9 shows the drug delivery device rotated by 90° about the longitudinal axis of the device compared with FIG. 10.

The compression spring 21 is designed and located so that it makes contact with the spring washer 7 of the pen injector mechanism during attachment of the cartridge holder 2. The compression spring 21 in the cartridge holder assembly provides a light force when the cartridge holder 2 is located in the blocked position with respect to an incompatible pen injector mechanism, sufficient only to move the cartridge holder 2 back up the bayonet track 3, away from the blocking location when the user releases their grip. In this example, the compression spring 21 in the cartridge holder assembly is not stiff enough to cause significant deflection of the spring washer 7 located in the pen mechanism.

FIG. 9 shows sectional views of the fourth embodiment, in which a cartridge holder 2 is located in the blocking position with respect to an incompatible pen injector mechanism. As can be seen in FIG. 9, the compression spring member 21 of the cartridge holder is slightly compressed by its' contact with the spring washer 7 of the pen injector under a user applied torque. When the user releases the cartridge holder 2 in this location, energy stored in the compression spring 21 by elastic deflection will be released, causing the cartridge holder 2 to unwind a small distance up the bayonet track 3.

If the cartridge holder 2 of the fourth embodiment is attached to a compatible pen injector mechanism, the compression spring member 21 will be deflected by contact with the spring washer 7 owing to its much lower stiffness. This means that the spring washer 7 is still able to perform its key function of engaging and biasing the cartridge 13 forwards onto its datum contact surface with the cartridge holder 2.

This is shown in FIG. 10, where the cartridge holder 2 has been correctly (fully) attached to a compatible mechanism housing 1 that is suitable for a 1.5 mL cartridge 13.

### REFERENCE NUMERALS

1 housing (inner body)
2, 2a-2g cartridge holder
3 bayonet track
4 bayonet protrusion
5 holder coding element (ramp)
6 housing coding element (ramp)
7 spring washer
8 outer housing
9 piston rod
10 driver
11 number sleeve
12 button
13 cartridge
14 clutch
15 pressure foot
16 rib
17 compliant region
18 slot
19 compliant region
20 slot
21 spring

1-15. (canceled)

16. A system for a drug delivery device comprising:
a housing comprising at least one housing coding element selected from a group of at least two different housing coding elements;
a cartridge holder comprising at least one holder coding element selected from a group of at least two different holder coding elements,
wherein the cartridge holder is releasably attachable to the housing when the housing coding element of the housing matches with the holder coding element of the cartridge holder, whereas a full attachment of the cartridge holder to the housing is prevented when the housing coding element of the housing does not match with the holder coding element of a cartridge holder,
wherein the cartridge holder is releasably attachable to the housing by at least one bayonet type attachment interface through an attachment movement comprising a first stage and a second stage, wherein the first stage comprises at least an axial movement component of the cartridge holder relative to the housing, and the second stage comprises at least a rotational movement component of the cartridge holder relative to the housing; and
at least one compliant spring member arranged and designed such that it is strained during the first stage and/or the second stage of the attachment movement at least when the housing coding element does not match the holder coding element,
wherein the compliant spring member is arranged and designed to move the cartridge holder relative to the housing in a direction contrary to the attachment movement when the compliant spring member is relaxing.

17. The system according to claim 16, wherein the at least one compliant spring member is a spring washer.

18. The system according to claim 17, wherein when a cartridge is retained in the cartridge holder:
the spring washer is arranged in the housing so as to exert a force on the cartridge, and
the cartridge holder is fully attached to the housing to secure the cartridge against an axial movement with respect to the cartridge holder.

19. The system according to claim 17, wherein the spring washer comprises fixing elements extending in an axial direction for fixing the spring washer within the housing.

20. The system according to claim 17, wherein at least one rib is formed on an inner wall of the cartridge holder,
wherein the at least one rib is located so that, when the housing coding element does not match the holder coding element, at least at an end of the first stage of the attachment movement, an end surface of the at least one rib engages and compresses the spring washer.

21. The system according to claim 20, wherein the at least one rib is located in the cartridge holder such that the at least one rib does not compress the spring washer when the housing coding element of the housing matches with the holder coding element of the cartridge holder.

22. The system according to claim 16, wherein the at least one compliant spring member comprises at least one compliant region of the cartridge holder which is, at least at an end of the first stage of the attachment movement, elastically deformable by an engagement of the housing coding element and the holder coding element when if the housing coding element does not match the holder coding element.

23. The system according to claim 22, wherein the at least one compliant region of the cartridge holder comprises an axially extending slot in the cartridge holder.

24. The system according to claim 22, wherein the at least one compliant region of the cartridge holder comprises a circumferentially extending slot in the cartridge holder.

25. The system according to claim 16, wherein the at least one compliant spring member comprises at least one compression spring having a distal end attached to or abutting the cartridge holder,
wherein at least at an end of the first stage of the attachment movement, the compression spring is elastically deformable by engagement of its opposite proximal end with the housing or with a component retained in the housing.

26. The system according to claim 25, wherein the at least one compression spring is configured as a metal wire-formed compression spring, a metal pressing, or a plastic molding.

27. The system according to claim 25, wherein at least at an end of the first stage of the attachment movement, the compression spring is elastically deformable by engagement of an opposite proximal end of the compression spring with a spring washer as the component is retained in the housing.

28. The system according to claim 27, wherein a stiffness of the spring washer is higher than a stiffness of the at least one compression spring.

29. The system according to claim 16, wherein the housing coding element and the holder coding element form a ramp interface, and wherein the second stage of the attachment movement is defined or governed by the ramp interface.

30. The system according to claim 16, wherein the bayonet type attachment interface is configured such that when the cartridge holder is attached to the housing, the cartridge holder is moved relative to the housing during the second stage of the attachment movement an axial distance in a second stage axial direction opposite to a direction of the axial movement component of the first stage.

31. The system according to claim 30, wherein the housing coding element and the holder coding element form a ramp interface, and
wherein matching of the housing coding element of the housing with the holder coding element of the cartridge holder is defined by a slope of the ramp interface being equal to a slope defined by the axial distance of the second stage.

32. A drug delivery device comprising:
a cartridge containing a medicament;
a housing comprising at least one housing coding element selected from a group of at least two different housing coding elements;
a cartridge holder holding the cartridge and comprising at least one holder coding element selected from a group of at least two different holder coding elements,
wherein the cartridge holder is releasably attachable to the housing when the housing coding element of the housing matches with the holder coding element of the cartridge holder, whereas a full attachment of the cartridge holder to the housing is prevented when the housing coding element of the housing does not match with the holder coding element of a cartridge holder,
wherein the cartridge holder is releasably attachable to the housing by at least one bayonet type attachment interface through an attachment movement comprising a first stage and a second stage, wherein the first stage comprises at least an axial movement component of the cartridge holder relative to the housing, and the second stage comprises at least a rotational movement component of the cartridge holder relative to the housing; and
at least one compliant spring member arranged and designed such that it is strained during the first stage and/or the second stage of the attachment movement at least when the housing coding element does not match the holder coding element,
wherein the compliant spring member is arranged and designed to move the cartridge holder relative to the housing in a direction contrary to the attachment movement when the spring member is relaxing.

33. The drug delivery device according to claim 32, wherein the at least one compliant spring member is a spring washer,
wherein the spring washer is arranged in the housing so as to exert a force on the cartridge, and
wherein the cartridge holder is fully attached to the housing to secure the cartridge against an axial movement with respect to the cartridge holder.

34. The drug delivery device according to claim 33, wherein at least one rib is formed on an inner wall of the cartridge holder,
wherein the at least one rib is located so that, when the housing coding element does not match the holder coding element, at least at an end of the first stage of the attachment movement, an end surface of the at least one rib engages and compresses the spring washer.

35. The drug delivery device according to claim 32, wherein the at least one compliant spring member comprises at least one compliant region of the cartridge holder which is, at least at an end of the first stage of the attachment movement, elastically deformable by an engagement of the housing coding element and the holder coding element when the housing coding element does not match the holder coding element.
